(19) United States
(12) Patent Application Publication
(10) Pub. No.: US 2023/0145684 A1
(43) Pub. Date: May 11, 2023

(54) MEDICAL DEVICE COMPRISING A LIQUID BALLOON FOR FORMING ARTIFICIAL SPHINCTERS

(71) Applicant: A.M.I. Agency for Medical Innovations GmbH, Feldkirch (AT)

(72) Inventors: Martin HOHLRIEDER, Götzis (AT); Roland HUBER, Mäder (AT)

(73) Assignee: A.M.I. Agency for Medical Innovations GmbH, Feldkirch (AT)

(21) Appl. No.: 17/918,355

(22) PCT Filed: Apr. 13, 2021
(86) PCT No.: PCT/EP2021/059480

§ 371 Date: Oct. 12, 2022

(30) Foreign Application Priority Data
Apr. 23, 2020 (AT) A 97/2020

(51) Int. Cl. A61F 2/00

(52) U.S. Cl. CPC A61F 2/004; A61F 2230/0065; A61F 2250/0013

(57) ABSTRACT

A device including a liquid balloon (1), a pump unit (5) connected to the liquid balloon (1) via a first connection line (3) for pumping liquid into the liquid balloon (1) and for pumping liquid out of the liquid balloon (1), an electric motor (6) for driving the pump unit (5), a battery (7) for supplying energy to the electric motor (6) and an electronic control unit (8) for controlling the electric motor (6). The device further includes a manual actuating unit (51) for removing liquid from the liquid balloon (1) and for pumping liquid back into the liquid balloon (1), the manual actuating unit (51) having a flexible pump body (30) which is connected to the liquid balloon (1) via a second connection line (31).

### INCORPORATION BY REFERENCE

The following documents are incorporated herein by reference as if fully set forth: PCT Patent Application No. PCT/EP2021/059480, filed Apr. 13, 2021; and Austrian Patent Application No. A 97/2020, filed Apr. 23, 2020.

### TECHNICAL FIELD

The invention relates to a device comprising a liquid balloon, a pump unit which is connected to the liquid balloon via a first connecting line for pumping liquid into the liquid balloon and for pumping liquid out of the liquid balloon, an electric motor for driving the pump unit, a battery for supplying power to the electric motor and an electronic control unit for controlling the electric motor.

### BACKGROUND

In the medical sector, liquid balloons which can be filled with a liquid and can be closed to form a ring are used to form artificial sphincters, amongst other things for the urethra. In order to close the artificial sphincter, liquid is pumped into the inner chamber of the liquid balloon which is closed to form a ring around a body passage to be blocked in order to expand the inner wall of the liquid balloon against the hollow organ to be blocked. In order to open the body passage, the liquid is emptied from the chamber of the liquid balloon.

Medical devices for narrowing or blocking off a body passage are also used elsewhere in the human body, for example for forming an artificial sphincter for an anus, possibly an artificial anus, as gastric bands for narrowing the gastrointestinal tract or as bands for closing a duct for bile. Medical devices of this kind are also referred to as cuffs, sleeves or artificial sphincters.

With a conventional design of an artificial sphincter, a manually operable pump with a flexible pump body is used for pumping liquid. In the case of the design as an artificial urethral sphincter, a manual operating device of this kind is implanted in the scrotum. Liquid is usually pumped out of the liquid balloon into a reservoir by applying pressure to the flexible pump body in order to clear the body passage. Following this, the body passage, e.g. the urethra, is automatically closed with a time delay and/or in a throttled manner due to liquid autonomously flowing back into the inner chamber of the liquid balloon, this being triggered, for example, by the pressure exerted by the elastically expanded reservoir.

US 2014/0364686 A1 discloses a plurality of exemplary embodiments of medical devices in the form of urethral sphincters which comprise a liquid balloon which is referred to as a cuff, wherein the cuff has an internal chamber and an external chamber. An intermediate wall, which separates the two chambers of the cuff from one another, has a throttle valve which allows liquid exchange between the chambers. In order to open the body channel, liquid is moved from the inner chamber to the outer chamber by means of a pump. The throttle valve allows the body passage to be automatically closed due to liquid gradually overflowing out of the outer chamber into the inner chamber.

U.S. Pat. No. 5,478,308 A discloses a medical device which can be used to treat urinary or fecal incontinence. The liquid balloon, which is also referred to as a cuff in said document, is produced from silicone. By filling the liquid balloon with fluid, the pressure in the cavity of the cuff increases, with the resulting displacement of an inner section of the cuff in the direction of an longitudinal centre axis closing the body passage. A flexible pump body is used to pump liquid into an elastically expandable storage container. Liquid is automatically returned to the cuff on account of the relative overpressure in the expanded storage container in relation to the pressure in the cuff via overflow ducts or throttle valves.

Furthermore, devices of the kind mentioned at the outset, in which a pump unit which is driven by an electric motor is present, have already been disclosed, wherein the electric motor is controlled by an electronic control unit. Such a device of the kind mentioned at the outset can be found in WO 2017/205883 A1. The pressure of the liquid in the receiving space of the pump unit is detected by a pressure sensor. In order to fill a liquid balloon placed annularly around a body passage and thereby block the body passage, liquid is pumped into the chamber of the liquid balloon by means of the pump unit. Here, the pressure is set to a specific target value by the control unit.

### SUMMARY

The object of the invention is to provide an advantageous device of the kind mentioned at the outset which has a high degree of operational reliability. This is achieved by a device having one or more of the features disclosed herein.

In addition to the motor-operated pump unit, a device according to the invention comprises a manual operating unit for removing liquid from the liquid balloon and for pumping liquid back into the liquid balloon, which manual operating unit has a flexible pump body which is connected to the liquid balloon via a second connecting line.

The provision of the manual operating unit in addition to the motorized pump unit ensures an increased degree of operational reliability. For example, the situation may in particular occur in practice that the user has forgotten the remote controller for operating the motorized operating unit. The manual operating unit may also be used in the event of the motorized operating unit not operating, for example if the battery has not been replaced in good time.

The manual operating unit preferably has a manually openable shut-off valve which is arranged between two sections of the second connecting line, wherein the two sections of the second connecting line are connected to one another in a liquid-conducting manner in an open position of the shut-off valve and the liquid-conducting connection between the two sections of the second connecting line is interrupted in a closed position of the shut-off valve. Here, liquid can advantageously be manually drained from the liquid balloon such that the shut-off valve is manually opened. Liquid can then flow out of the liquid balloon into the flexible pump body. The volume of a pump chamber which is arranged within the pump body is increased in size in the process, preferably without the material of the pump body expanding, i.e. the pump body is only folded apart (not for example elastically expanded) when liquid flows in.

In a preferred embodiment of the invention, the manually openable shut-off valve is opened and, with the shut-off valve open, the pump body is compressed in order to manually pump liquid into the liquid balloon. Therefore, operation by the user can be performed using both hands.

The motorized pump unit advantageously has a receiving space which can be filled with liquid and the volume of which can be changed by moving an actuating part of the pump unit by means of the electric motor. Furthermore, there is expediently a pressure sensor for detecting the pressure of the liquid in the receiving space. It is preferred that the detected pressure of the liquid in the receiving space of the motorized pump unit is stored in the control unit after completion of a respective actuating process of the actuating part that was executed by means of the electronic control unit and is compared with the pressure currently prevailing in the receiving space before carrying out a further actuating process. If a magnitude of a change in pressure which lies above a specified threshold value is established by the control unit here, this means there is a malfunction. For example, liquid could have been manually drained from the liquid balloon since the last motorized actuating process, without having been pumped back into the liquid balloon again. Therefore, a request to the user to manually pump liquid into the liquid balloon can be output, for example, on a remote controller of the device. If the drop in pressure cannot be eliminated in this way, it is concluded that there is a leak. The instruction that an urgent medical consultation be made can then be output on the remote controller for the user.

A device according to the invention can advantageously form an artificial sphincter, in particular a urethral sphincter, wherein the liquid balloon is in the form of a flexible band which can be closed to form a ring and has a longitudinally running inner chamber which can be filled with liquid.

### BRIEF DESCRIPTION OF THE DRAWINGS

Further advantages and details of the invention are explained below with reference to the attached drawing, in which:

FIG. 1 shows a device according to the invention in the implanted state;

FIGS. 2 and 3 show a side view and view of the motorized operating unit;

FIG. 4 shows a section along the line AA from FIG. 3 in a first position of the actuating part;

FIG. 5 shows a section corresponding to FIG. 4 in a second position of the actuating part;

FIG. 6 shows an exploded illustration;

FIG. 7 shows an oblique view of the plate of the travel sensor which is electrically connected to the electronic control unit and has the spiral conductor track;

FIG. 8 shows an oblique view of the bellows of the pump unit;

FIG. 9 shows an oblique view of the liquid balloon which is closed to form a ring;

FIG. 10 shows an axial view of the liquid balloon which is closed to form a ring in the unfilled state of the inner chamber;

FIG. 11 shows a section along the line BB from FIG. 10;

FIG. 12 shows an illustration corresponding to FIG. 10 in the filled state of the inner chamber;

FIG. 13 shows a side view of the manual operating unit;

FIG. 14 shows a plan view of the manual operating unit;

FIG. 15 shows a section along the line CC from FIG. 13 in the non-operated state of the manual operating unit;

FIG. 16 shows a section analogous to FIG. 15 in the operated state of the manual operating unit;

FIG. 17 shows a section along the line DD from FIG. 14 in the closed state of the shut-off valve;

FIG. 18 shows a section corresponding to FIG. 17 in the open state of the shut-off valve; and

FIG. 19 shows an exploded illustration of the manual operating unit.

### DETAILED DESCRIPTION

An exemplary embodiment of a device according to the invention is illustrated in the figures.

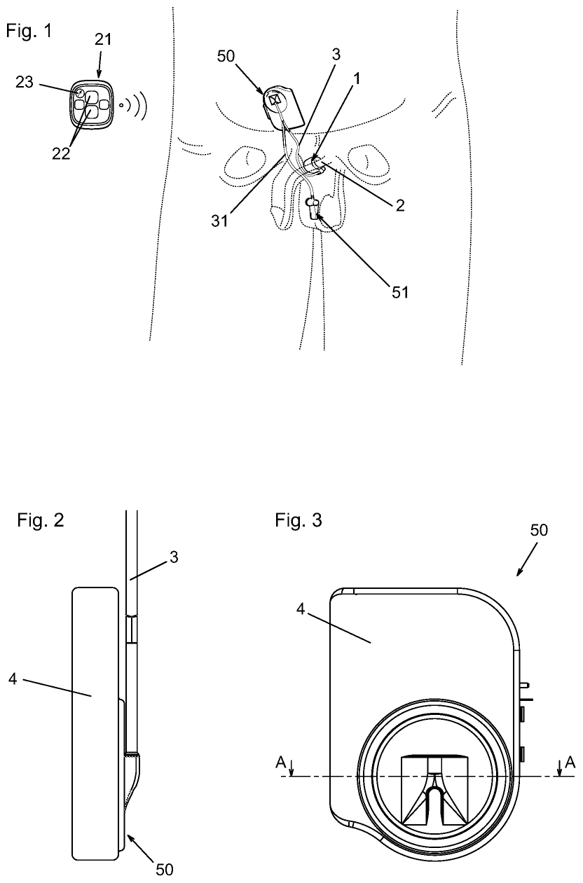

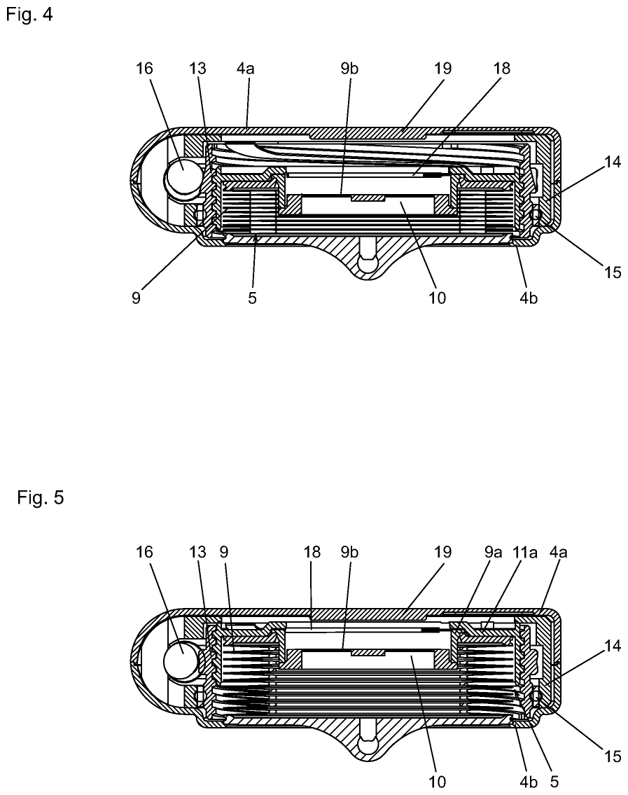

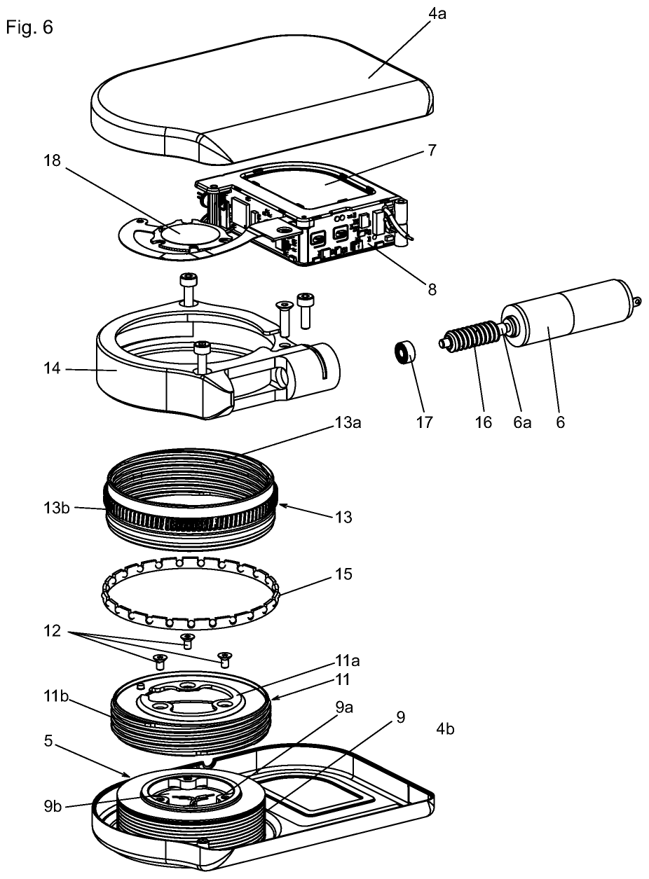

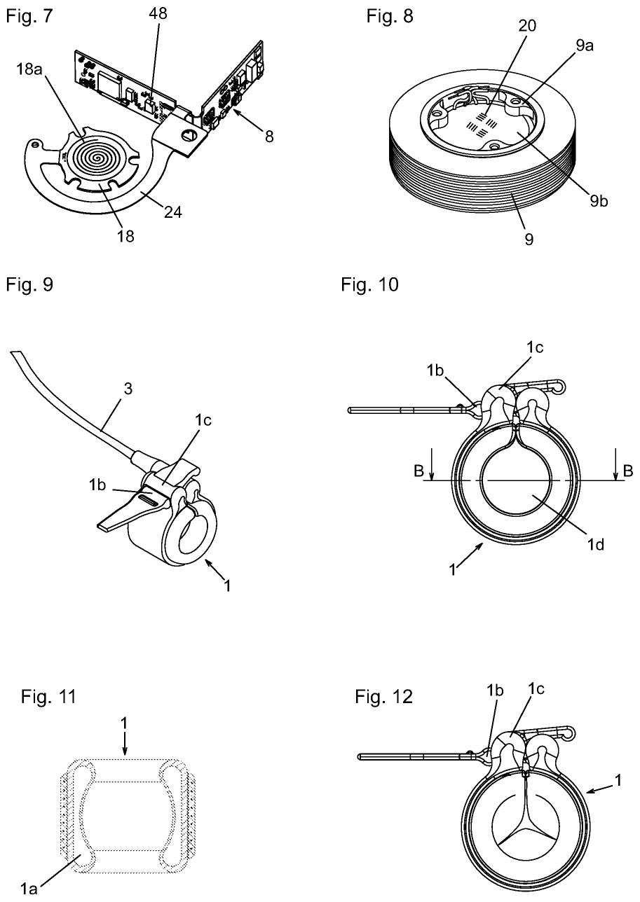

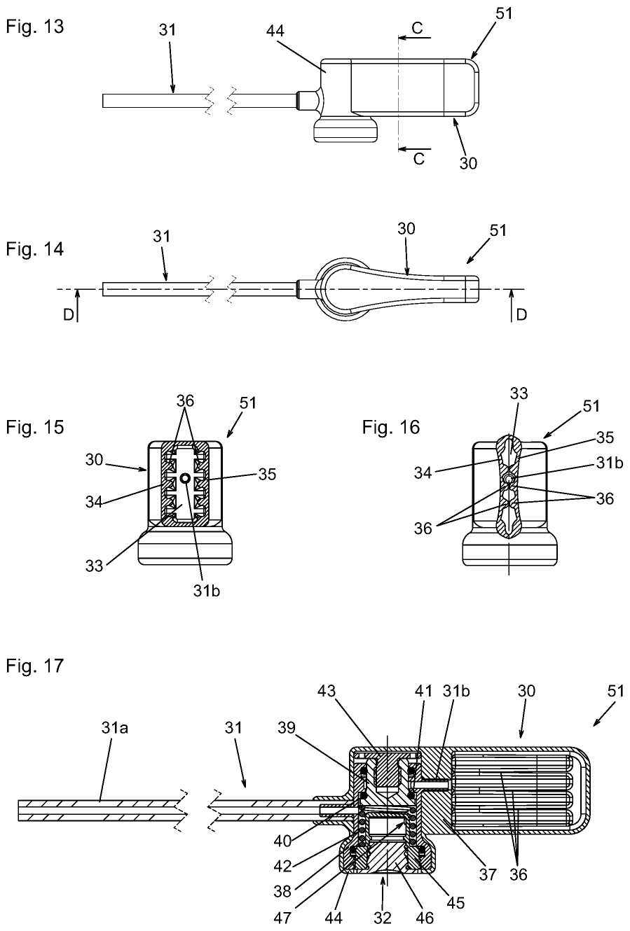

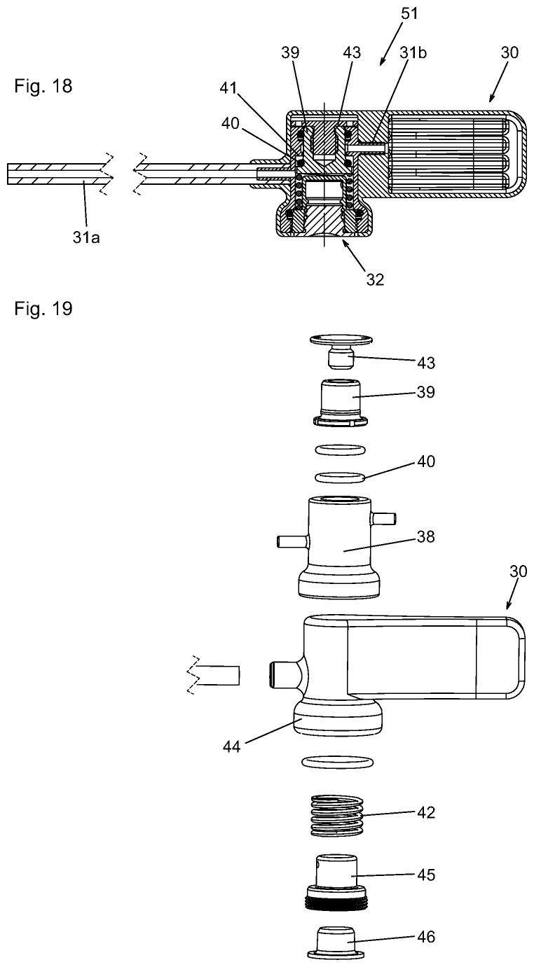

A liquid balloon 1 is designed in the form of a flexible band having a longitudinally running inner chamber 1a. The liquid balloon can be closed to form a ring by means of closure parts 1b, 1c which are arranged at the two end regions, wherein said liquid balloon can be placed in a ring shape around a body passage, here the urethra 2, cf. FIG. 1.

When the liquid balloon 1 is closed to form a ring and the chamber 1a is emptied, the liquid balloon 1 which is closed to form a ring has a passage opening 1d, cf. FIG. 11. The size of the passage opening 1d can be reduced by pumping liquid into the chamber 1a, cf. FIGS. 10 and 12.

A pump unit 5, which is connected to the liquid balloon 1 via a first connecting line 3 (which is configured as a hose) and is arranged in a motorized operating unit 50 which can be implanted into the body, is used for pumping liquid into the liquid balloon 1 and for pumping liquid out of the liquid balloon 1. This motorized operating unit 50 has a housing 4 in which, apart from the pump unit 5, an electric motor 6 for driving the pump unit 5, a battery 7 for supplying power to the electric motor 6 and an electronic control unit 8, which controls the electric motor 6 and therefore also the pump unit 5 and is likewise fed by the battery 7, are arranged.

In the exemplary embodiment, the housing 4 comprises a half-shell-shaped upper housing part 4a and a half-shell-shaped lower housing part 4b which are connected to one another in a gas-tight manner. The interior of the housing 4 is therefore insulated from the area surrounding the housing 4 in a gas-tight manner.

The pump unit 5 has a bellows 9, in particular folding bellows. The bellows 9 encloses a receiving space 10 in which a liquid, e.g. saline solution, is located. Furthermore, the pump unit 5 has an actuating part 11 which is rigidly connected to an end piece 9a of the bellows, in the exemplary embodiment by means of screws 12. The actuating part 11 has a bottom 11a which is connected to the end piece 9a of the bellows 9 and has a sleeve section which surrounds the outside of the bellows 9 and has an external thread 11b. An internal thread 13a of an actuating ring 13 is in engagement with the external thread 11b. The actuating ring 13 is rotatably mounted in a bearing housing 14. A ball bearing 15 can be provided for rotatably mounting the actuating ring 13 in the bearing housing 14, in particular as illustrated. The actuating ring 13 also has a toothing 13b which surrounds the outside of said actuating ring. A worm gear 16 which is driven by the electric motor 6 is in engagement with the toothing 13b of the actuating ring. The worm gear 16 can be arranged directly on the motor shaft 6a of the electric motor 6.

In the exemplary embodiment, the electric motor 6 is held on the bearing housing 14 and the motor shaft 6a is mounted at the end side by a bearing ring 17 such that it can rotate with respect to the bearing housing 14.

Strain gauges are fitted, for example sputtered, onto the end membrane 9b of the bellows 9, as indicated in FIG. 8. These strain gauges provide a pressure sensor 20 for detecting the pressure which acts between the receiving space 10 of the bellows 9 and the space surrounding the bellows 9 (=interior of the housing 4). Pressure sensors of this kind which are formed by means of strain gauges are known per se.

The pressure sensor 20 is electrically connected to the control unit 8.

A plate 18 which has a spiral conductor track 18a is rigidly connected to the actuating part 11. In the exemplary embodiment, the plate 18 is arranged between the actuating part 11 and the end piece 9a of the bellows 9. The plate 18 is used as a sensor for the distance from the section 19 of the wall of the housing 4, which section is situated opposite the plate. Therefore, an inductive travel sensor for detecting the position of the actuating part 11 is formed by the plate 18 in connection with the section 19 of the wall of the housing 4.

The conductor track 18a of the plate 18 is electrically connected to the control unit 8. In the exemplary embodiment, a flexible conductor track carrier 24 which runs from the plate 18 in the form of a helicoidal surface to the control unit 8 is provided for this purpose.

When the electric motor 6 is operated by the control unit 8, this electric motor adjusts the actuating part 11 in the axial direction of the helical gear mechanism and the bellows 9 via the worm gear mechanism, which is formed between the electric motor and the actuating ring 13, and the helical gear mechanism, which is formed between the actuating ring 13 and the actuating part 11. As a result, the volume of the receiving space 10 is changed.

A remote control arrangement 21 is preferably provided for operating the device. Wireless data transmission takes place between the remote control arrangement 21 and the control unit 8. The remote control arrangement 21 has operator control elements 22. These operator control elements can be used to switch over between an open state and a closed state of the device. A first fill level of the liquid balloon 1 with a first filling pressure prevails in the open state and a differently filled second fill level of the liquid balloon with a second filling pressure which is higher than at the first fill level prevails in the closed state.

An air pressure sensor 23 for detecting the atmospheric pressure pU is also arranged in the remote control arrangement 21, the importance of which will be explained in more detail further below.

In order to detect the air pressure pG in the hermetically sealed housing 4, a housing air pressure sensor 48 is arranged in the housing 4, the importance of which will be explained in more detail further below.

In particular, it can be provided that the liquid balloon 1 is intended to assume only two different states which correspond to an open state and a closed state of the device, wherein the filling pressure in the liquid balloon in the closed state of the device is greater than the filling pressure in the open state.

In order to set a desired fill level of the liquid balloon 1, the electric motor 6 is operated in a corresponding manner by the control unit 8. For example, to this end, the control unit 8 can carry out corresponding pressure control by way of the pressure in the receiving space 10 of the bellows 9 being set to the desired value and on the basis of the value which is output by the pressure sensor 20 (wherein the system is allowed to relax to a sufficient extent before terminating the setting operation in order to create pressure equalization). The travel sensor 18, 19 could then be dispensed with too.

On the other hand, it could be provided that travel control of the actuating part 11 is carried out by the control unit 8 for setting a desired fill level of the liquid balloon 1 with a desired end position as the target position. This travel control is advantageously carried out as discontinuous control, preferably as two-point control with the control values “motor on” and “motor off”.

With this travel control, the actual position of the control part 11 is continuously detected by means of the travel sensor 18, 19 and initially the control value “motor on” is sent to the electric motor 6. The desired end position of the actuating part 11 minus a specified latency distance is set as the switch-off position. When the actual position of the actuating part 11 reaches this switch-off position, the electric motor 6 is switched off. The travel control can then be terminated. However, after the electric motor 6 has been switched off, a check can also be made as to whether the desired target position has been reached within a specified tolerance range and, if not, the latency distance for subsequent travel control operations can be adapted and/or the position of the actuating part 11 can be adjusted, so that the pressure p in the receiving space lies within the permissible filling pressure range after the position of the actuating part 11 has been adjusted.

In order to carry out the travel control, a learning cycle for determining a characteristic curve which shows the relationship between the position of the actuating part and the pressure of the liquid in the receiving space of the pump device is preferably carried out when the device is put into operation for the first time or else repeatedly. This characteristic curve is preferably set as a straight line, wherein the value of the gradient k of the characteristic curve is stored by the control unit 8. If, in the learning cycle, that is to say when recording the characteristic curve, more than two points are approached, the straight lines can be optimized (for example to the smallest sum of the squares of the errors).

In order to determine the pressure of the liquid in the receiving space 10 of the pump unit 5, the air pressure in the housing 4 and a change in the atmospheric pressure are taken into account. The pressure sensor 20 detects the differential pressure between the two sides of the end membrane 9b of the bellows 9. Since the atmospheric pressure pU acts on the liquid balloon 1 and therefore also on the liquid contained in the liquid balloon, the pressure acting on the end membrane 9b on the liquid side is the sum of the liquid pressure p and the atmospheric pressure pU. The air pressure pG prevailing within the housing 4 is applied to the opposite side of the end membrane 9b. Since the housing 4 is hermetically sealed, this air pressure changes only as a function of the volume which the bellows 9 occupies in its respective position, that is to say in the respective position of the actuating part 11. The pressure value pS output by the pressure sensor 20 is therefore:

pS=p+pU−pG.

In order to determine the pressure of the liquid in the receiving space 10 of the pump units 5, the atmospheric pressure pU which is output by the air pressure sensor 23 is therefore subtracted from the pressure value pS which the pressure sensor 20 outputs, and the air pressure pG in the interior of the housing (and outside the bellows) which is output by the housing air pressure sensor 48 is added.

In addition to the motorized operating unit 50, a manual operating unit 51 is also provided, with which a manual changeover can be made between the open state and the closed state of the device, in particular if the user were to forget the remote controller 21. The manual operating unit 51 will be explained in more detail in the text which follows.

The manual operating unit 51 has a flexible pump body 30 which is connected to the liquid balloon 1 via a second connecting line 31. Furthermore, the manual operating unit 51 has a manually openable shut-off valve 32 which is arranged between two sections 31a, 31b of the connecting line 31. In an open position of the shut-off valve 32, the two sections 31a, 31b of the second connecting line are connected to one another in a liquid-conducting manner. In a closed position of the shut-off valve 32, the liquid-conducting connection between the two sections 31a, 31b is interrupted.

The first section 31a of the second connecting line 31, which first section is formed by a hose, connects the shut-off valve 32 to the inner chamber 1a of the liquid balloon 1. The second section 31b of the second connecting line 31, which second section is substantially shorter than the first section 31a, connects the shut-off valve 32 to a pump chamber 33 which is arranged in the pump body 30.

The first connecting line 3 and the second connecting line 31 could be connected to the liquid balloon 1 separately and open out into the chamber 1a of the liquid balloon 1 at separate points. In the exemplary embodiment however, a common line section, which splits into the first connecting line 3 and the second connecting line 31, exits from the liquid balloon 1.

The volume of the pump chamber 33 which is arranged in the pump body 30 can be reduced by an operating force which can be manually exerted by the user and acts on the pump body 30. For this purpose, in particular, two opposite side walls 34, 35 of the pump body 30 can be pressed together until they are brought into contact. At least one of the side walls 34, 35, preferably both side walls 34, 35, has/have a plurality of raised portions 36 on their side situated in the interior of the pump chamber 33. When the side walls 34, 35 are in a state in which they are fully pressed together, they rest against one another via the raised portions 36. The raised portions may be, in particular, fins which are spaced apart from one another and preferably run in parallel.

The second section 31b of the second connecting line 31, which second section runs through a connecting wall 37 of the pump body, which connecting wall connects the opposite side walls 34, 35, opens into the pump chamber 33 at a point which, in the state in which the opposite side walls 34, 35 are fully pressed together, lies in the region of a recess which is located between or next to the raised portions 36 of the at least one side wall 34, 35. In a sectional view orthogonal to the direction of longitudinal extent of the pump body 30, the second connecting line 31 opens into the pump chamber 33 in the region between two raised portions 36 of the same side wall 34, 35. As a result, when the side walls 34, 35 are pressed together until the side walls 34, 35 come into contact with one another, liquid can flow out of the pump chamber 33 (with the shut-off valve 32 open).

It would also be conceivable and possible for the raised portions 36 to end at a distance from the connecting wall 37.

The shut-off valve 32 has a closure member 39 which is displaceably mounted in a valve housing 38. Starting from a closed position, in which the closure member 39 is sealed off from a valve seat 41 by means of a seat seal 40, the closure member 39 can be displaced against the force of a return spring 42 into an open position. For this purpose, an operating button 43 which is connected to the closure member 39 or formed in one piece with it is pressed in.

The return spring 42 is supported on a closure part 45 which is screwed into the valve housing 38. The closure part is of pot-shaped design and sealed off from the surrounding area by a port 46. The port 46 is used to introduce liquid into the device by piercing it with a cannula. Openings 47 are made in the pot-shaped closure part 45 here.

The shut-off valve 32 and the pump body 30 are advantageously formed integrally with one another. This integral unit also comprises the second section 31b of the connecting line 31.

A sheath 44 which sheaths the shut-off valve 32 and is formed in one piece with the pump body 30 is preferably present.

In order to manually empty the liquid balloon 1, the shut-off valve 32 is opened by pressing the operating button 43, as a result of which liquid can flow out of the chamber 1a of the liquid balloon into the pump chamber 33 of the pump body 30.

In order to fill liquid into the chamber 1a of the liquid balloon 1, the shut-off valve 32 is opened by pressing the operating button 43 and the side walls 34, 35 of the pump body 30 are pressed together until they make contact with one another. After the operating button 43 is released, liquid is blocked from flowing back out of the chamber 1a of the liquid balloon 1.

It would also be conceivable and possible for the shut-off valve 32 to be of self-opening design when the pump body 30 is compressed, for example by way of appropriately dimensioning the return spring 42.

When designed as an artificial urethral sphincter, the manual operating unit 51 is preferably implanted in the scrotum.

The pressure p of the liquid prevailing in the receiving space 10 of the pump unit 5 is preferably stored after each actuating process which is carried out by the control unit 8. Before a renewed actuating process, a check is made as to whether the current pressure p of the liquid in the receiving space 10 of the pump unit 5 is still within a tolerance range around the stored value of the pressure p. If this is not the case, the reasons for this may essentially be as follows:

Liquid could have been drained from the liquid balloon 1, which was filled with liquid during the last motorized actuating process, by means of the manual operating unit 51. If the liquid is then pumped back into the liquid balloon 1 by means of the manual operating unit 51, the pressure p of the liquid in the receiving space 10 can be checked once again. If it is not possible to achieve the situation that the pressure p lies in the specified range in this way, it is assumed that there is a leak and therefore the device needs to be checked.

### LEGEND FOR THE REFERENCE NUMERALS

1 Liquid balloon
1a Chamber
1b Closure part
1c Closure part
1d Passage opening
2 Urethra
3 First connecting line
4 Housing
4a Upper housing part
4b Lower housing part
5 Pump unit
6 Electric motor
6a Motor shaft
7 Battery
8 Control unit
9 Bellows
9a End piece
9b End membrane
10 Receiving space
11 Actuating part
11a Bottom
11b External thread
12 Screw
13 Actuating ring
13a Internal thread
13b Toothing
14 Bearing housing
15 Ball bearing
16 Worm gear
17 Bearing ring
18 Plate
18a Conductor track
19 Section
20 Pressure sensor
21 Remote control arrangement
22 Operator control element
23 Air pressure sensor
24 Conductor track carrier
30 Pump body
31 Second connecting line
31a First section
31b Second section
32 Shut-off valve
33 Pump chamber
34 Side wall
35 Side wall
36 Raised portion
37 Connecting wall
38 Valve housing
39 Closure member
40 Seat seal
41 Valve seat
42 Return spring
43 Operating button
44 Sheath
45 Closure part
46 Port
47 Opening
48 Housing air pressure sensor
50 Motorized operating unit
51 Manual operating unit

1. A device comprising:
a liquid balloon;
a pump unit connected to the liquid balloon via a first connecting line for pumping liquid into the liquid balloon and for pumping liquid out of the liquid balloons;
an electric motor for driving the pump unit;
a battery for supplying power to the electric motor;
an electronic control unit configured to control the electric motor; and
a manual operating unit for removing liquid from the liquid balloon and for pumping liquid back into the liquid balloon, the manual operating unit including a flexible pump body which is connected to the liquid balloon via a second connecting line.

2. The device as claimed in claim 1, wherein the motor-operated pump unit has a receiving space that is fillable with liquid and a volume of which is changeable by moving an actuating part of the pump unit using the electric motor.

3. The device as claimed in claim 2, wherein the pump unit has a bellows within which the receiving space for the liquid is situated, and an end piece of the bellows is rigidly connected to the actuating part.

4. The device as claimed in claim 2, further comprising at least one pressure sensor for detecting a pressure of the liquid in the receiving space.

5. The device as claimed in claim 4, wherein the pressure sensor has at least one strain gauge attached to an end membrane which delimits an interior of the bellows in an axial direction of the bellows.

6. The device as claimed in claim 5, further comprising an air pressure sensor configured to detect atmospheric pressure and a housing air pressure sensor configured to detect an air pressure in a gas-tight-insulated interior of a housing in which the pump unit is arranged, and the electronic control unit is configured for determining a pressure of the liquid in the receiving space of the pump unit by subtracting the atmospheric pressure which is detected by the air pressure sensor from a measured value which is output by the pressure sensor and adding the air pressure in the housing which is detected by the housing air pressure sensor.

7. The device as claimed in claim 1, further comprising a travel sensor for detecting the position of the actuating part.

8. The device as claimed in claim 1, wherein the manual operating unit further comprises a manually openable shut-off valve which is arranged between two sections of the second connecting line, wherein the two sections of the second connecting line are connected to one another in a liquid-conducting manner in an open position of the shut-off valve and the liquid-conducting connection between the two sections of the second connecting line is interrupted in a closed position of the shut-off valve.

9. The device as claimed in claim 8, wherein the shut-off valve has a return spring which acts on a closure member of the shut off valve and presses the closure member against a sealing seat of the shut-off valve in the closed position.

10. The device as claimed in claim 8, wherein the flexible pump body has a pump chamber for receiving liquid which is supplied to the pump body via the second connecting line, the pump chamber is delimited by two mutually opposite side walls of the pump body, and the side walls are movable into contact so as to reduce a volume of the pump chamber.

11. The device as claimed in claim 10, wherein at least one of the opposite side walls has a plurality of raised portions on a side situated in an interior of the pump chamber.

12. The device as claimed in claim 1, wherein the device forms an artificial sphincter, the liquid balloon comprises a flexible band which is closable to form a ring and has a longitudinally running inner chamber which is fillable with the liquid, and a passage opening of the liquid balloon which is closed to form a ring is reducible in size by reducing a volume of the pump body.

13. A method for operating a device as claimed in claim 2, the method comprising storing a pressure of the liquid in the receiving space of the motorized pump unit in the control unit after completion of a respective actuating process of the actuating part that was executed by the electronic control unit and comparing the stored pressure with a pressure currently prevailing in the receiving space before carrying out a further actuating process.

14. The method as claimed in claim 13, wherein the actuating process comprises travel control of the actuating part for approaching a target position of the actuating part, including detecting a respective position of the actuating part using a travel sensor, and, after the travel control, checking whether the pressure of the liquid in the receiving space of the motor-operated pump unit lies within a permissible filling pressure range around the second filling pressure, and if the pressure of the liquid in the receiving space lies outside the permissible filling pressure range around the second filling pressure, adjusting the position of the actuating part.

15. The method as claimed in claim 13, further comprising storing a characteristic curve in the control unit that shows a relationship between a position of the actuating part and the pressure of the liquid in the receiving space, and using the characteristic curve for determining a target position of the actuating part, and the characteristic curve is a straight line with a gradient stored by the control unit.
